(19) United States
(12) Patent Application Publication
(10) Pub. No.: US 2024/0032783 A1
(43) Pub. Date: Feb. 1, 2024

(54) ENDOSCOPE DEVICE

(71) Applicant: ClearMind Biomedical, Inc., San Jose, CA (US)

(72) Inventors: Sheng-Chi LIN, New Taipei City (TW); Feng-Cheng CHANG, New Taipei City (TW); Yu-Jen LIN, New Taipei City (TW)

(21) Appl. No.: 18/359,454

(22) Filed: Jul. 26, 2023

Related U.S. Application Data
(60) Provisional application No. 63/394,085, filed on Aug. 1, 2022.

(51) Int. Cl. A61B 1/12; A61B 1/05; A61B 1/00

(52) U.S. Cl. CPC A61B 1/126; A61B 1/05; A61B 1/00094; A61B 2017/320024

(57) ABSTRACT

An exemplary endoscope may be provided with a first shaft, a second shaft and an image capture module. The first shaft may extend from a distal end of an endoscope body and may include a distal opening and a proximal opening. The second shaft may extend through an interior of the first shaft and may also include a distal opening and a proximal opening. The image capture module may be in operable communication with the endoscope body distal end. The endoscope may be configured to clear debris from the image capture module with suction when the second shaft proximal opening and first shaft proximal opening are in alignment. Methods of use are also disclosed.

### CLAIM OF PRIORITY

This application claims the benefit of U.S. Provisional Patent Application No. 63/394,085, filed Aug. 1, 2022, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention relates generally to endoscope devices. More particularly, the present invention provides systems and methods for endoscopic procedures including a cleaning system for an image module of an endoscope.

### BACKGROUND

Endoscopes are commonly used for minimally or non-invasive medical procedures and can support various aspects of the procedure such as modifying tissues by cutting, grinding, and imaging. Endoscope devices often combine debridement capability with imaging modules to support navigation and operation of associated debridement mechanisms. During endoscopic procedures various bodily tissue, liquids and or other materials, debris or pollutants can contact the imaging modules and stain, contaminate, or otherwise obstruct the lens. Accordingly, the loss of imaging can result in extended procedure times, increased potential for unintentional damage to non-target tissues and more.

Attempts to address this issue often involve directing a flow of fluid towards the image module with additional structural elements coupled to the endoscope. The size of the additional structures facilitating the flow of fluid increases the overall size of the endoscope and inhibits opportunities for minimally invasive procedures. Additionally, the flow of fluid requires substantial modification of an endoscope to facilitate fluid reservoir, routing of the fluid and subsequent removal of the fluid. Beyond the diminution of the minimally invasive capability of these systems, clogged nozzles or ports associated with the flow of fluid can render existing clearing systems inactive.

For these reasons, it would be desirable to provide improved methods, systems, and devices for clearing endoscope imaging modules. It would be particularly desirable to provide simplified debridement systems and assemblies having fewer components and, even more desirably, to provide components that can effectively clear and maintain endoscope imaging capability while also improving the function of the endoscope in modifying tissue during a procedure. At least some of these objectives are met by the various embodiments that follow.

### SUMMARY OF THE DISCLOSURE

Described herein are endoscope devices, and clearing systems for removing debris from camera modules associated with the endoscope device.

In general, an endoscope can comprise a first shaft extending from a distal end of an endoscope body, the first shaft comprising a distal opening and a proximal opening; a second shaft extending through an interior of the first shaft, the second shaft comprising a distal opening and a proximal opening; and an image capture module in operable communication with the endoscope body distal end, the endoscope configured to clear debris from the image capture module with suction when the second shaft proximal opening and first shaft proximal opening are in alignment.

This and any example described herein may also comprise one or more of the following: A second shaft distal end can be configured to cut tissue extending through the first shaft distal opening. The endoscope can be configured to alternate suction between the second shaft distal opening and the second shaft proximal opening.

In some embodiments, the second shaft distal opening comprises a perimeter configured to cut tissue extending into the second shaft. The second shaft distal opening may comprise an inclined perimeter from a proximal side of the second shaft distal opening to a distal side of the proximal shaft distal opening. The first shaft distal opening and second shaft distal opening can be configured to be in alignment when the first shaft proximal opening and second shaft proximal opening are mis-aligned. The first shaft distal opening and second shaft distal opening can be configured to be mis-aligned when the first shaft proximal opening and second shaft proximal opening are aligned. The first shaft comprises a closed distal end. The second shaft can be configured to rotate in the first shaft. The second shaft comprises a lumen configured to direct suction supplied to the second shaft distal opening and second shaft proximal opening. The second shaft can be configured to oscillate in the first shaft. The second shaft distal opening can be the distal end of the second shaft. The second shaft can be configured to oscillate between a distal position and a proximal position. The second shaft proximal opening can be positioned on a first side of the second shaft, and the second shaft distal opening can be positioned on a second side of the second shaft opposite the first side. The second shaft distal opening and second shaft proximal opening can be positioned on the same side of the second shaft. The second shaft can be configured to reciprocate within the first shaft. The second shaft can be configured to alternate rotation from a first direction to a second direction. The first shaft distal opening can be positioned on a side of the first shaft, and wherein the second shaft distal opening defines a perimeter of a distal end of the second shaft.

In general, an endoscope can comprise an endoscope body with a distal end comprising an image capture module; an outer tube extending distally from the endoscope body, the outer tube comprising a proximal opening and a distal opening; and an inner tube within the outer tube, the inner tube comprising a proximal opening configured to direct suction to the outer tube distal opening when aligned with the inner tube proximal opening.

In general, an endoscope image module clearing system an comprise a first shaft extending distally from an endoscope body, the first shaft comprising a distal opening and a proximal opening positioned proximal to an image capture module in a distal end of the endoscope body; and a second shaft configured to oscillate within the first shaft, the second shaft comprising a distal opening configured to cut tissue, and a proximal opening configured to direct suction to the first shaft proximal opening when aligned with the second shaft proximal opening.

In general, an endoscope image module clearing system can comprise an inner shaft configured to rotate within an outer shaft, the inner shaft and outer shaft extending from a distal end of an endoscope body; and an image capture module locatable at a distal end of the endoscope body, wherein the outer shaft comprises an opening adjacent to the image capture module, wherein the inner shaft comprises a proximal opening configured to be selectively aligned with the outer shaft proximal opening to direct suction to the image capture module.

In general, a method of cleaning an image capture module of an endoscope can comprise moving an inner shaft extending distally from an endoscope body, the inner shaft locatable within an interior of an outer shaft; then aligning a proximal opening of the inner shaft with a proximal opening of the outer shaft; then supplying suction from the inner shaft through the inner shaft proximal opening and the outer shaft proximal opening, wherein the suction is configured to clear debris/liquid from the image capture module.

In general, a method of cleaning an image capture module of an endoscope during an endoscopic procedure can comprise oscillating an inner shaft extending distally from an endoscope body, the inner shaft locatable within an interior of an outer shaft; then suctioning tissue into the outer shaft through an outer shaft distal opening; then cutting the tissue with edges of the inner shaft distal opening when the inner shaft distal opening moves relative to the outer shaft distal opening; then supplying suction from the inner shaft, when a proximal opening of the inner shaft is aligned with a proximal opening of the outer shaft, wherein the supplied suction is configured to clear debris/liquid from the image capture module.

In general, a method of cleaning an image capture module of an endoscope during an endoscopic procedure can comprise reciprocating an inner shaft extending distally from an endoscope body, the inner shaft locatable within an interior of an outer shaft; then alternating alignment of an inner shaft proximal opening with an outer shaft proximal opening; and then supplying suction from the inner shaft proximal opening to the outer shaft proximal opening, when the inner shaft proximal opening is aligned with the outer shaft proximal opening, wherein the suction is configured to clear debris from an image capture module.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A is a perspective view of an endoscope showing an example of an endoscope device distal end as described herein.

FIG. 1B is an exploded diagram showing an example of an endoscope device as described herein.

FIG. 1C is a side elevation view of an endoscope showing an example of an endoscope device distal end as described herein.

FIG. 1D is a perspective view of an endoscope showing an example of an endoscope distal end as described herein.

FIG. 2A is a perspective view of an endoscope showing an example of an endoscope distal end as described herein.

FIG. 2B is a side elevation view of an endoscope showing an example of an endoscope distal end as described herein.

FIG. 2C is a side elevation view of an endoscope showing an example of an endoscope distal end as described herein.

FIG. 2D is a is a perspective view of an endoscope showing an example of an endoscope distal end as described herein.

FIG. 3A is a perspective view of an endoscope showing an example of an endoscope distal end as described herein.

FIG. 3B is a perspective view of an endoscope showing an example of an endoscope distal end as described herein.

FIG. 3C is a side elevation view of an endoscope showing an example of an endoscope distal end as described herein.

FIG. 3D is a side elevation view of an endoscope showing an example of an endoscope distal end as described herein.

FIG. 3E is a perspective view of an endoscope showing an example of an endoscope distal end as described herein.

FIG. 4A is an expanded view of an endoscope showing details of a distal end example as described herein.

FIG. 4B is a perspective view showing an example of a second shaft of an endoscope device as described herein.

FIG. 5A is an expanded view of an endoscope showing details of a distal end example as described herein.

FIG. 5B is a cross-sectional showing an example of an endoscope with details of a distal end as described herein.

FIG. 5C is a cross-sectional showing an example of an endoscope with details of a distal end as described herein.

FIG. 6A is a perspective view of an endoscope showing an example of an endoscope distal end as described herein.

FIG. 6B is a side elevation view showing an example of an endoscope device as described herein.

FIG. 6C is a perspective view of an endoscope showing an example of an endoscope distal end as described herein.

FIG. 7A is a perspective view of an endoscope showing an example of an endoscope distal end as described herein.

FIG. 7B is a perspective view of an endoscope showing an example of an endoscope distal end as described herein.

FIG. 7C is a perspective view of an endoscope showing an example of an endoscope distal end as described herein.

FIG. 8 is a flow diagram illustrating an example of a method of using an endoscope device as described herein.

### DETAILED DESCRIPTION

An endoscope device may have an elongate body with a distal segment having a first shaft and second shaft extending beyond a distal end of the elongate body. The second shaft can be configured to move within the first shaft. The first shaft may have a proximal end and a distal end with one or more openings positioned between the first shaft proximal end and first shaft distal end. The second shaft may have a proximal end and a distal end with one or more openings positioned between the first shaft proximal end and the first shaft distal end. Openings on the first shaft and the second shaft may be locatable on each shaft such that an opening on the second shaft can be configured to align with and opening on the first shaft.

In some examples, the endoscope device can have an imaging element such as an image capture module (e.g., camera module). The camera module can be in operable communication with the endoscope device and configured to capture images, video, etc. In some examples, the camera module may be positioned at the distal end of the elongate body (e.g., endoscope body distal end) to facilitate visualization of the position and operation of the endoscope device. A proximal opening on the first shaft and second shaft may be positioned near the endoscope body distal end and adjacent to the cameral module.

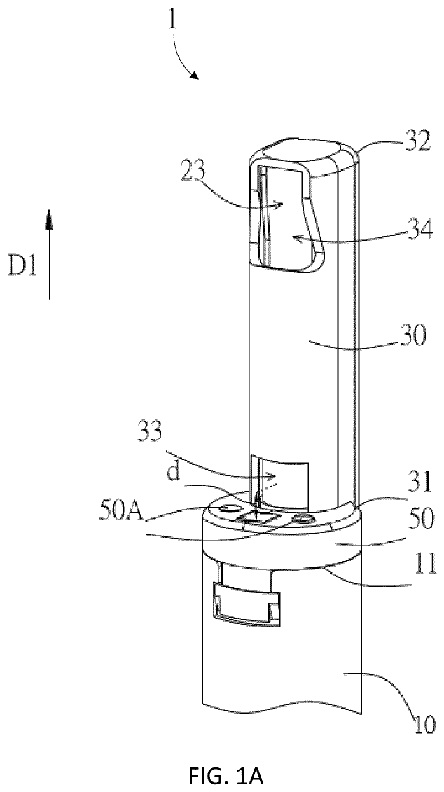

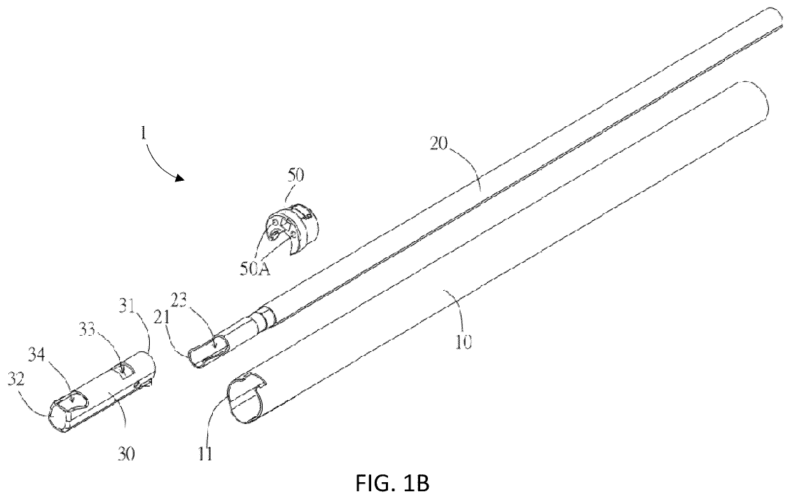

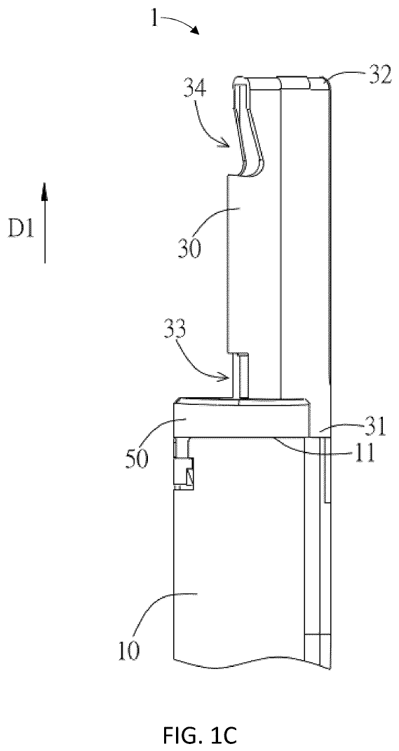

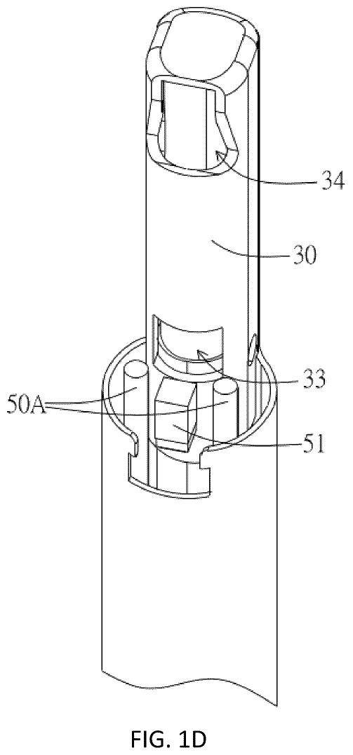

FIGS. 1A-1C respectively illustrate a three-dimensional schematic diagram, an exploded diagram, and side schematic diagram of endoscope device examples as described herein. As illustrated in FIGS. 1A-1C, the endoscope device 1 comprises an endoscope body 10, a second shaft 20, and a first shaft 30. The endoscope body 10 has a body distal end 11 with the second shaft 20 extending from the body distal end 11 to the second shaft distal end 21. The first shaft 30 is configured to at least partially cover the second shaft 20, and has a first end 31 and a second end 32 opposite to each other along a first direction D1. In this example, the second end 32 is provided corresponding to the second shaft distal end 21 (i.e., the first end 31 is a proximal end, and the second end 32 is a distal end). However, the present invention is not limited thereto.

In the present embodiment, a first ring 50 containing a camera module can be provided on the body distal end 11. An endoscope lens 50A is provided on the first ring 50. The shape of the first ring 50, can be configured to cover the endoscope body. For example, the first ring can be a semicircle configured to cover portions of the endoscope body 10 and/or the first shaft 30. Referring to FIG. 1D, the body distal end 11 is visible with the first ring 50 removed. This view illustrates an interior of the endoscope body 10 with an example of a camera module 51 of the endoscope device 1 after removing the first ring 50. As mentioned above, the endoscope device 1 can contain a camera module 51 including the endoscope lens 50A in the first ring 50 (not shown in FIG. 1D), and the camera module 51 can be of different shapes such as square, circular, or rectangle and the like. However, sizes and shapes of the first ring 50 and the camera module 51 of the present invention are not limited thereto. FIG. 1D also shows an example of the positioning of the first shaft proximal opening relative to the camera module 51.

As illustrated in FIGS. 1A-1C, the first shaft 30 has a proximal opening 33 near the proximal end 31 and the body distal end 11, and a first shaft distal opening 34 near the second end 32 (e.g., first shaft distal end) and the second shaft distal end 21. The characteristics of each opening on the first shaft and second shaft may comprise a size, shape, orientation, arrangement, or other attributes configured to facilitate the operation of the endoscope device described herein.

In some examples, diameters (e.g., size) of the first shaft proximal opening 33 and the first shaft distal opening 34 are not limited; however, on a virtual cross-section of the first shaft 30, areas of the first shaft proximal opening 33 and the first shaft distal opening 34 respectively surrounding the first shaft 30 can be less than or equal to 180 degrees of a central angle. In some examples, the areas of the first shaft proximal opening 33 and the first shaft distal opening 34 respectively surrounding the first shaft 30 are preferably greater than or equal to 180 degrees of a central angle. In some examples, sizes of the second shaft proximal opening 22 and the second shaft distal opening 23 are not limited; however, on a virtual cross-section of the second shaft 20, areas of the second shaft proximal opening 22 and the second shaft distal opening 23 respectively surrounding the second shaft 20 can be less than or equal to 180 degrees of a central angle. In some examples, the areas of the second shaft proximal opening 22 and the second shaft distal opening 23 respectively surrounding the second shaft 20 are preferably greater than or equal to 180 degrees of a central angle.

In some examples, the size of the first shaft distal opening 34 can be larger than that of the first shaft proximal opening 33. For example, the size of the first shaft distal opening 34 may be equal to or larger than five times the size of the first shaft proximal opening 33. In some examples, the first shaft proximal opening 33 is collinear with the first shaft distal opening 34 along a first direction D1. For example, the first shaft proximal and distal openings may be located at the same side of the first shaft 30. However, the present invention is not limited thereto. In some examples, the first shaft proximal opening and first shaft distal opening may be non-collinear with each other along the first direction D1 (e.g., offset relative to one another). In some examples, the size of the second shaft distal opening 23 can be larger than that of the second shaft proximal opening 22. For example, the size of the second shaft distal opening 23 may be equal to or larger than five times the size of the second shaft proximal opening 22. In some examples, the second shaft proximal opening 22 is collinear with the second shaft distal opening 23 along a first direction D1. For example, the second shaft proximal and distal openings may be located at the same side of the second shaft 20. In some examples, the second shaft proximal opening and second shaft distal opening may be non-collinear with each other along the second direction D1 (e.g., offset relative to one another).

In some examples, the first shaft proximal opening 33 and first shaft distal opening 34 may be some distance from one another along the first shaft 30. For example, the distance between the center of the first shaft proximal opening 33 and the center of the first shaft distal opening 34 can be 1.5 cm, less than 1.5 cm, greater than 1.5 cm, etc.; however, the distance between the first shaft proximal opening 33 and the first shaft distal opening 34 is not limited thereto. In some examples, the second shaft proximal opening 22 and second shaft distal opening 23 may be some distance from one another along the second shaft 20. For example, the distance between the center of the second shaft proximal opening 22 and the center of the second shaft distal opening 23 can be 1.5 cm, less than 1.5 cm, greater than 1.5 cm, etc.; however, the distance between the second shaft proximal opening 22 and the second shaft distal opening 23 is not limited thereto. In some examples, the distance between the first shaft proximal opening and first shaft distal opening may be equal to, less than, or greater than the distance between the second shaft proximal opening 22 and the second shaft distal opening 23.

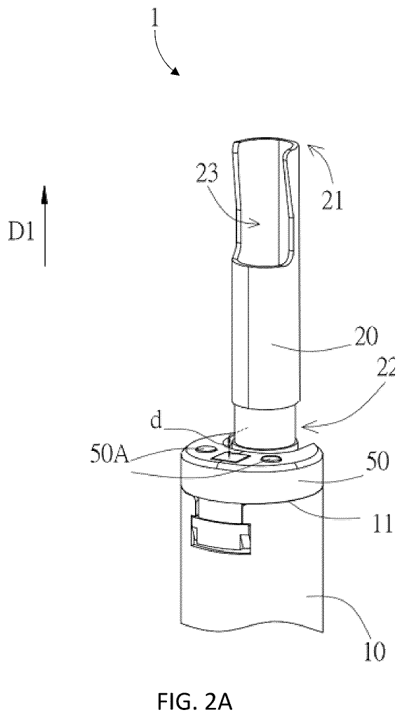

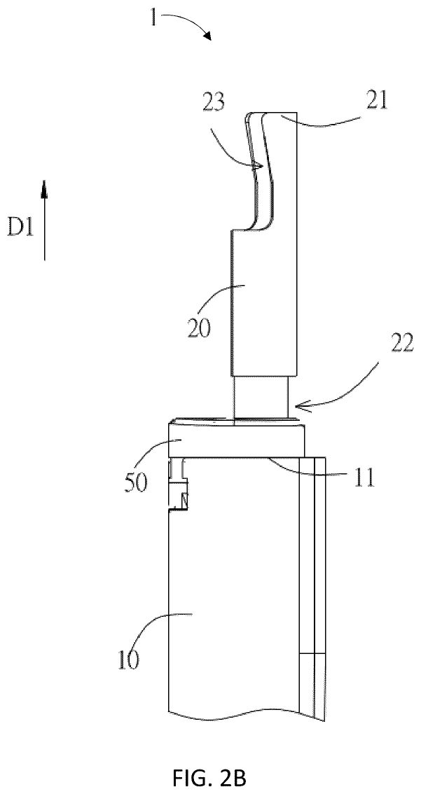

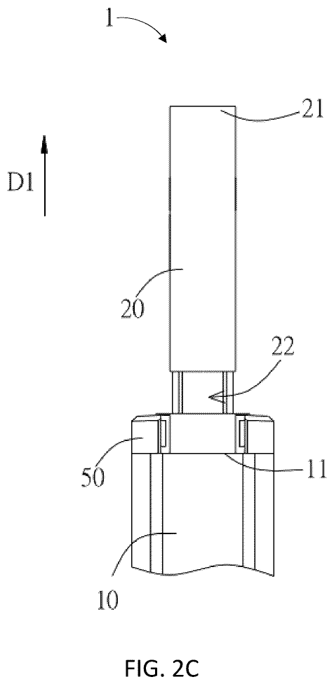

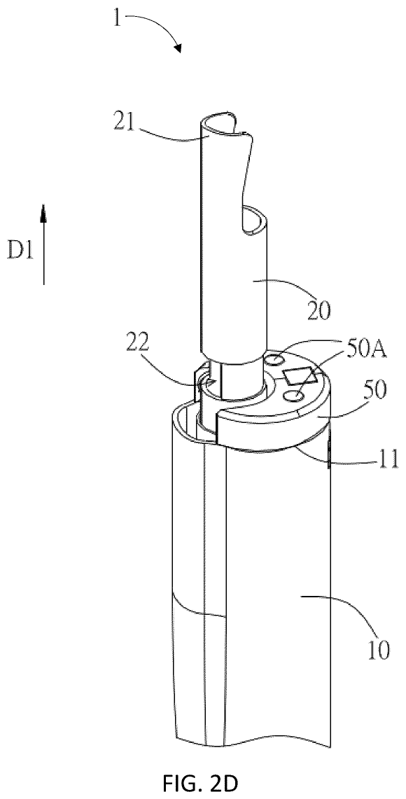

FIGS. 2A-2D, illustrate a three-dimensional schematic diagram, a side diagram, a back diagram, and a three-dimensional schematic diagram, respectively, from another angle of the endoscope device 1 with the first shaft 30 removed to expose details of the second shaft 20. As illustrated in FIGS. 2A-2D, the second shaft 20 has a second shaft proximal opening 22 near the body distal end 11 and a second shaft distal opening 23 near the second shaft distal end 21. In the present embodiment, the second shaft proximal opening 22 and the second shaft distal opening 23 are respectively located on different side surfaces of the second shaft 20. For example, when the second shaft proximal opening 22 is located on a first side (not illustrated) of the second shaft 20, the second shaft distal opening 23 is located on a second side (not illustrated) of the second shaft 20 opposite to the first side. In this example, when the second shaft distal opening 23 and the first shaft distal opening 34 are aligned and communicated with each other (e.g., as illustrated in FIG. 1A), the second shaft proximal opening 22 and the first shaft proximal opening 33 are not aligned and communicated with each other. In this example, a preferred angle between the center position of the second shaft proximal opening 22 and the center position of the first shaft proximal opening 33 may be 180 degrees. For example, the second shaft's proximal opening 22 and the first shaft's proximal opening 33 are positioned on opposite sides of the second shaft 20. For instance, when the first shaft's proximal opening 33 is facing upwards, the second shaft's proximal opening 22 is facing downwards. However, as long as the second shaft proximal opening 22 and the second shaft distal opening 23 are located on different sides of the second shaft 20, the present invention is not limited thereto.

In some examples, the diameter (size) of the first shaft distal opening 34 is larger than the diameter (size) of the first shaft proximal opening 33 (e.g., as illustrated in FIG. 1C). When the second shaft distal opening 23 and the first shaft distal opening 34 are aligned with each other (as illustrated in FIG. 1A), and suction is supplied to the distal end of the endoscope (e.g., second shaft distal end), the majority of suction may be directed to the distal end of the first and second shafts. In some examples, the sizes of the second shaft proximal opening 22 and the second shaft distal opening 23 are not limited. For example, on a virtual cross-section of the second shaft 20, areas of the second shaft proximal opening 22 and the second shaft distal opening 23 surrounding the second shaft 20 are preferably and respectively less than or equal to 180 degrees of a central angle. Accordingly, when the second shaft distal opening 23 and the first shaft distal opening 34 being located on different planes, it can be ensured that the second shaft proximal opening 22 and the first shaft proximal opening 33 are mis-aligned with each other when the second shaft distal opening 23 and the first shaft distal opening 34 are aligned and communicated with each other (as illustrated in FIG. 1A); For example, alignment of the first and second shaft proximal opening may alternate relative to the first and second shaft distal openings as the second shaft moves within the first shaft. In some examples, sizes of the openings can be suitably adjusted in accordance with the actual location of each opening. In addition, in the present embodiment, the size of the first shaft distal opening 34 can be the same as that of the second shaft distal opening 23 or smaller than that of the second shaft distal opening 23.

The second shaft 20 can be configured to move relative to the first shaft 30. Movement of the second shaft 20 (e.g., within the first shaft 30) may be rotational, lateral, or a combination thereof. For example, the second shaft 20 may be rotatable within the first shaft 30. Rotation of the second shaft can provide selective alignment between the openings of the first and second shafts. For example, the second shaft 20 may be axially rotated within the first shaft 30 whereby the first shaft proximal opening 33 and the second shaft proximal opening 22 are aligned at some period of the rotation of the second shaft 20. Similarly, rotation of the second shaft 20 may provide for alignment of the first shaft distal opening 34 and the second shaft distal opening 23.

In some examples, (e.g., as illustrated in FIGS. 1B, 2A and 2B) the second shaft distal opening 23 may have an inclined plane and be concaved with respect to the second shaft 20. These and other characteristics of the second shaft distal opening can provide a cutting function of the second shaft distal opening 23. For example, when the second shaft 20 is being rotated, an edge portion of the second shaft distal opening 23 configured to cut, sever, macerate, etc. tissue. In some examples, (e.g., as illustrated in FIGS. 1A, 1B, and 1D) the first shaft distal opening 34 can also have an inclined plane and be concaved with respect to the first shaft 30. As such, when the second shaft distal opening 23 and the first shaft distal opening 34 are aligned and communicated with each other (e.g., as illustrated in FIG. 1A), the endoscope body 10 can be configured to suction liquid via the aligned and communicated second shaft distal opening 23 and the first shaft distal opening 34 or to operate on the object (e.g., the tissues of the patient) using the second shaft distal end 21. Wherein a relative movement (e.g., rotatory motion, lateral motion, reciprocation, back and forth motion, oscillating motion, etc.) between the second shaft 20 and the first shaft 30 can change the relative position between the second shaft distal opening 23 and the first shaft distal opening 34 and the relative position between the second shaft proximal opening 22 and the first shaft proximal opening 33. In addition, the use when the second shaft distal opening 23 and the first shaft distal opening 34 are aligned and communicated with each other is different from that when the second shaft proximal opening 22 and the first shaft proximal opening 33 are aligned and communicated with each other. The specific examples of application will be described later.

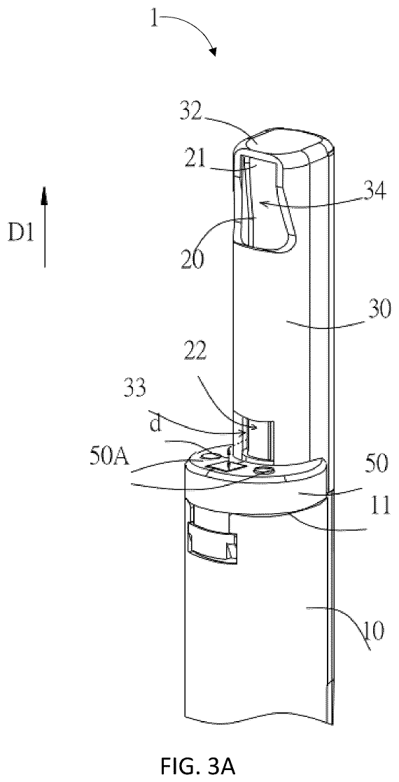

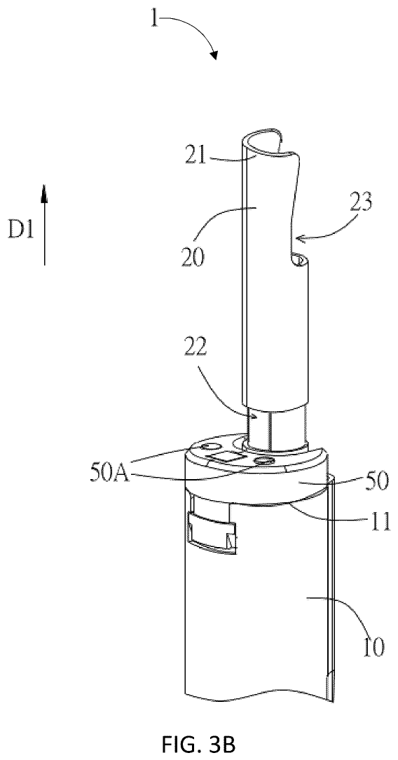

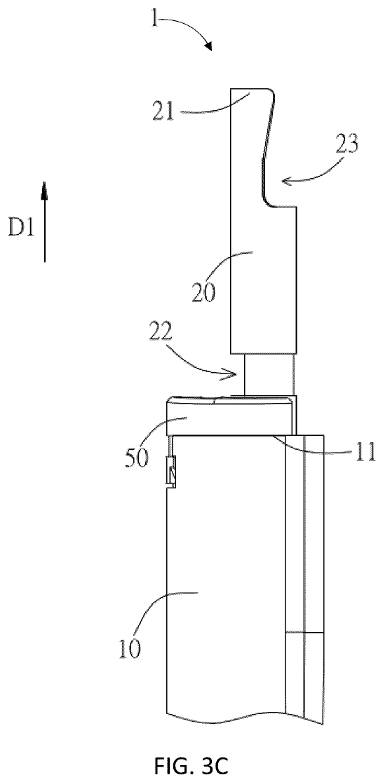

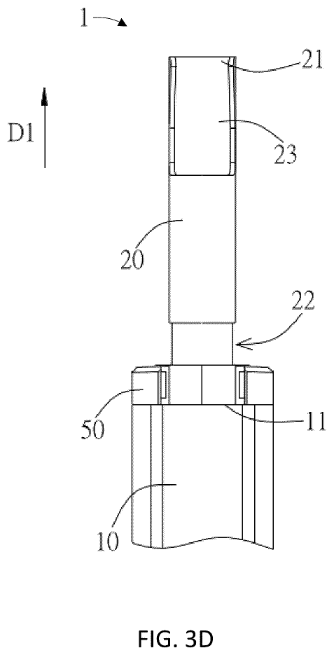

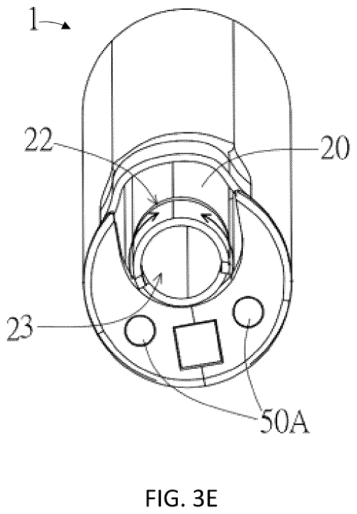

FIG. 3A is a three-dimensional schematic diagram of an endoscope device, such as the endoscope device illustrated in FIG. 1A when the second shaft 20 is rotated by 180 degrees. FIG. 3B illustrates a three-dimensional schematic diagram of the embodiment illustrated in FIG. 3A after removing the first shaft 30. FIGS. 3C-3E respectively illustrate a side diagram, a back diagram, a three-dimensional schematic diagram viewed from another direction, and a front diagram viewed from the second shaft distal end of the embodiment illustrated in FIG. 3B (that is, FIGS. 3B-3D respectively illustrate the state of the embodiment illustrated in FIGS. 2A-2D after rotating the second shaft 20 by 180 degrees). As illustrated in FIGS. 3A-3B, the second shaft 20 can change the relative position between the second shaft proximal opening 22 and the first shaft proximal opening 33 to make the second shaft proximal opening 22 and the first shaft proximal opening 33 aligned and communicated with each other by rotation (e.g., using a motor installed in the endoscope body 10). In that case, the second shaft distal opening 23 and the first shaft distal opening 34 are not completely aligned and communicated with each other, and at least a portion of the first shaft distal opening 34 is covered by the second shaft 20 (e.g., the second shaft distal opening 23 is opposite to the first shaft distal opening 34). However, the present invention is not limited thereto. In the different embodiments, when the second shaft distal opening 23 and the first shaft distal opening 34 are aligned and communicated with each other, the second shaft proximal opening 22 and the first shaft proximal opening 33 can be partially communicated with each other. Alternately, when the second shaft proximal opening 22 and the first shaft proximal opening 33 are aligned and communicated with each other, the second shaft distal opening 23 and the first shaft distal opening 34 can also be partially communicated with each other. During the time of interaction, two sets of openings can be kept partially aligned and communicated with each other. For example, the distance between the second shaft proximal opening 22 and the body distal end 11 may be approximately same as that between the first shaft proximal opening 33 and the body distal end 11, so that in the process of rotating the second shaft 20, the second shaft proximal opening 22 and the first shaft proximal opening 33 are aligned and communicated with each other. In some examples, the thickness of the wall of the second shaft 20 near the second shaft proximal opening 22 can be smaller relative to the second wall of other portions (i.e., the diameter is smaller than that of the other portions). For example, when the second shaft proximal opening 22 is not aligned and communicated with the first shaft proximal opening 33, the liquid transported via the second shaft 20 can be blown out from the first shaft proximal opening 33 along the paths when suction is not applied in FIG. 3E (i.e., surrounding the gap between the second shaft 20 and the first shaft 30). Accordingly, the liquid in the back side can be transported to the front side within the second shaft 20, the cut tissues in the front side or the pollutants suctioned from the first shaft proximal opening 33 can also be transferred to the back side within the second shaft 20. Therefore, it can be used for washing the endoscope lens 50A on the first ring 50. When the second shaft proximal opening 22 is aligned and communicated with the first shaft proximal opening 33, debris (e.g., tissue, liquid, etc.) on the endoscope lens 50A could be removed by using suction via the second shaft. Suction, as described herein, can be configured to clear the debris from the camera module. In some examples, the diameter (size) of the second shaft proximal opening 22 can be the same as the diameter (size) of the first shaft proximal opening 33 or can be larger than or less than the diameter (size) of the first shaft proximal opening 33.

Through such a configuration, the endoscope body 10 of the present invention is configured to perform debridement (cut the targeted object) and suction out the pollutants on the lens simultaneously based on the rotation of the second shaft 20. As such, for example, in the process of the surgery, decreased visibility due to polluted endoscope lens 50A can be avoided.

In some examples, along the first direction D1, the distance between the second shaft proximal opening 22 and the endoscope lens 50A can be the same as that between the first shaft proximal opening 33 and the endoscope lens 50A. In some examples, (e.g., as illustrated in FIG. 3A), the first shaft proximal opening 33 can be close to the second end 32 compared with the endoscope lens 50A, and the distance d therebetween may be less than 1 mm, 1 mm, or greater than 1 mm. For example, the distance d may be less than or equal to 3 mm. In some examples, the first shaft proximal opening 33 can be further from the second end 32 compared to the endoscope lens 50A (i.e., the first shaft proximal opening 33 is aligned with the first ring 50), and the distance there between may be less than equal to, or greater than 1.0 mm. For example, by having the first shaft proximal opening 33 located within the range (when the direction toward the second end 32 is viewed as a positive direction, the first shaft proximal opening 33 is located within the range of −1.0 mm to 3.0 mm with the endoscope lens 50A as a center), the efficiency of the endoscope body 10 washing the endoscope lens 50A or suctioning pollutants on the endoscope lens 50A can be enhanced. In any example, the function, position, characteristics, etc. of the first shaft proximal opening 33 and the second shaft proximal opening 22 being close to the first end 31 and the body distal end 11 are not limited to washing the endoscope lens 50A or suctioning the pollutants on the endoscope lens 50A, and the distance between the first shaft proximal opening 33 and the body distal end 11 can be adjusted in accordance with the actual uses.

In some examples, when the second shaft proximal opening 22 and the first shaft proximal opening 33 are aligned and communicated with each other (as illustrated in FIG. 3A), the angle between the center positions of the second shaft distal opening 23 and the first shaft distal opening 34 is 180 degrees, so that the second shaft distal opening 23 and the first shaft distal opening 34 respectively face different sides (e.g., when the first shaft distal opening 34 faces upward, the second shaft distal opening 23 faces downward). However, the angle between the second shaft distal opening 23 and the first shaft distal opening 34 is not limited to 180 degrees.

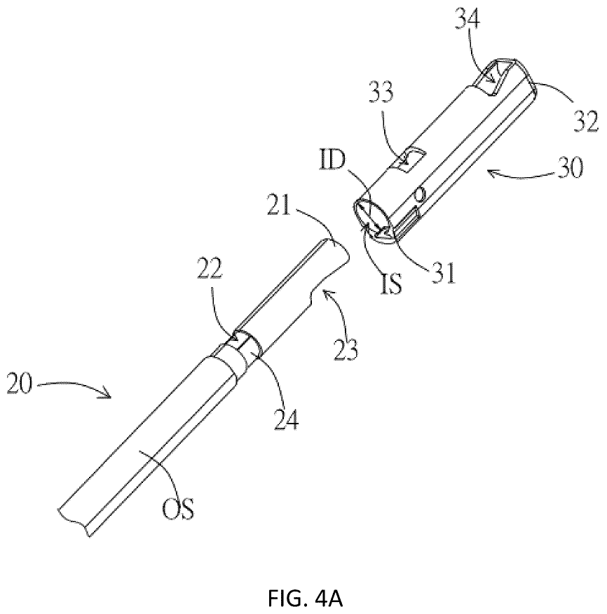

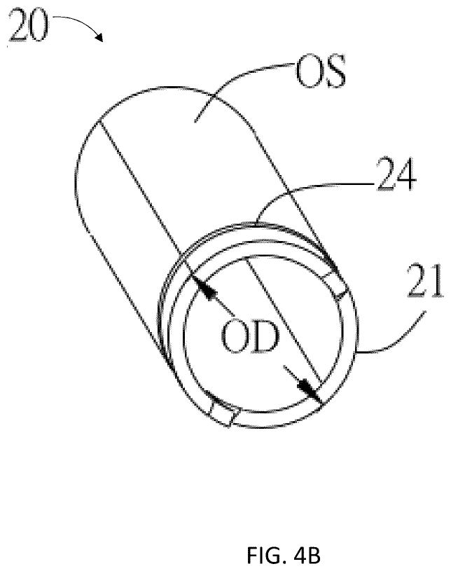

FIGS. 4A and 4B illustrate a three-dimensional diagram of the second shaft 20 and the first shaft 30 in an embodiment of the endoscope device 1 of the present invention, FIG. 4B illustrates a top view of the second shaft 20 in an embodiment of the endoscope device 1 of the present invention viewed from the second shaft distal end 21. In some examples, an inner diameter of the first shaft 30, as indicated by “ID” in FIG. 4A, may be the same, or greater than the outer diameter of the second shaft 20, as indicated by “OD” in FIG. 4B). For example, when the first shaft 30 covers the second shaft 20, the exterior surface of the second shaft 2, as indicated by “OS” in FIG. 4A adjacent to, in contact with, or a combination thereof relative to the interior surface, as indicated by “IS” in FIG. 4A, of the first shaft 30. In some examples, as illustrated in FIG. 4A, the exterior surface (e.g., OS) of second shaft 20 may have a concaved (e.g., grooved) region 24, and the second shaft proximal opening 22 is located on the grooved region 24. The grooved region 24, for example, may be an annular channel around a perimeter of the second shaft 20. Accordingly, the concaved region 24 can have a smaller diameter compared to other regions of the second shaft 20. This concave region 24 can be configured to facilitate liquid flow when the second shaft proximal opening 22 is not aligned and communicated with the first shaft proximal opening 33. For example, liquid transported via the first shaft 30 can flow out from the first shaft proximal opening 33 along the groove.

In some examples, the second shaft 20 is configured to be rotated to change the relative position between the second shaft proximal opening 22 and the first shaft proximal opening 33 and the relative position between the second shaft distal opening 23 and the first shaft distal opening 34. However, in a varied embodiment of the present invention, the relative movement between the second shaft 20 and the first shaft 30 is not limited to rotational motion.

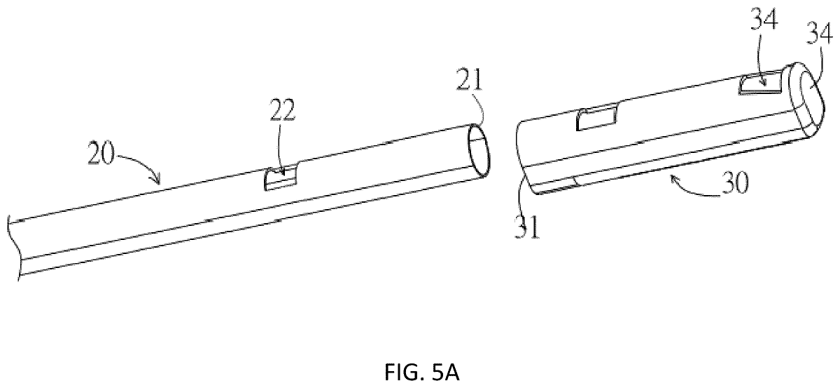

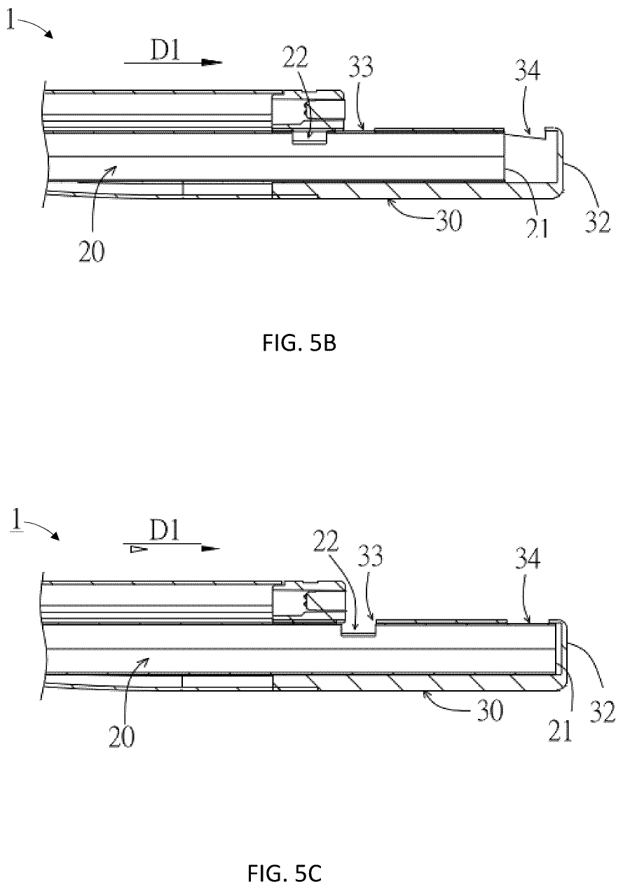

FIGS. 5A, 5B, and 5C, illustrate an example of an endoscope device where the second shaft 20 and the first shaft 30 are depicted. FIG. 5A presents a three-dimensional schematic diagram, while FIGS. 5B and 5C provide cross-sectional schematic diagrams of the second shaft 20 at different locations. In some examples, the second shaft 20 may be configured to move linearly (e.g., laterally) along the first direction D1 (radial direction of the second shaft 20) between a first position (e.g., illustrated in FIG. 5B) and a second position (e.g., illustrated in FIG. 5C). This movement alters the relative positioning of the second shaft's proximal opening 22 and the first shaft's proximal opening 33, as well as switches the first shaft's distal opening 34 between an open and covered state. The distal end/opening 21 of the second shaft can be utilized for cutting purposes and similar functions. In this example, the second shaft 20 may not have a distal opening on a side of the second shaft. In some examples, the second shaft distal opening may be the distal end of the second shaft such that the distal end of the second shaft is an aperture or opening (e.g., 21 in FIG. 5A) with a plane generally perpendicular to a plane of the second shaft proximal opening. In such a configuration, the lateral movement (e.g., oscillation, reciprocation, advancement) within the first shaft 30 can cut tissue extending through the first shaft distal opening. For example, tissue may extend through the first shaft distal opening 34 and when the second shaft 20 advances distally within the first shaft 30, the open distal end of the second shaft 30 can cut the tissue extending through the first shaft distal opening 34. Alignment between the second shaft proximal opening 22 and first shaft proximal opening 33 can continue to alternate alignment based on the lateral movement and position of the second shaft 20 within the first shaft 30.

In FIG. 5B, the position is set for preparation of cutting. The first shaft's distal opening 34 is open to allow suction of tissues or liquids, while the first shaft's proximal opening 33 remains closed. For example, when the second shaft proximal opening 22 and the first shaft proximal opening 33 are not in alignment, increased suction can be directed to the distal end of the second shaft (e.g., 21 illustrated in FIG. 5B). In FIG. 5C, the second shaft 20 is moving forward to cover the first shaft's distal opening 34. Additionally, the first shaft's proximal opening 33 and second shaft's proximal opening 22 are aligned and communicated providing for suction of tissues or liquid in front of the endoscope lens.

In some examples, when the second shaft 20 is positioned at the first location (as shown in FIG. 5B), the distal end 21 of the second shaft does not cover the distal opening 34 of the first shaft. Additionally, the proximal opening 22 of the second shaft and the proximal opening 33 of the first shaft are not aligned or connected, resulting in them being staggered from each other. In this configuration, the endoscope body 10 can be configured to perform suctioning of tissues or liquids through the open distal opening 34 of the first shaft. Then, when the second shaft 20 is situated at the second location (as depicted in FIG. 5C), the proximal opening 22 of the second shaft and the proximal opening 33 of the first shaft align and establish communication allowing for washing the endoscope lens or suctioning debris, pollutants and other material obstructing the camera module.

In some examples, when the first shaft's proximal opening 33 and the first shaft's distal opening 34 are staggered in their open states through relative movement, such as rotational or linear motion along the radial direction of the second shaft 20, the resulting continuous and/or alternating suctioning is configured to clean of the endoscope lens based on the movement of the second shaft 20. This allows for the removal (e.g., suctioning) of obstructions to the camera module, with continued adequate suction power at the distal end. Accordingly, reduced visibility caused by pollutants during endoscope usage can be minimized.

In some examples, there may be a time delay or difference between the opening and communication of the first shaft's proximal opening 33 and the first shaft's distal opening 34, the relative movement between the second shaft 20 and the first shaft 30, as well as the configurations of each opening, are not restricted to the aforementioned embodiments.

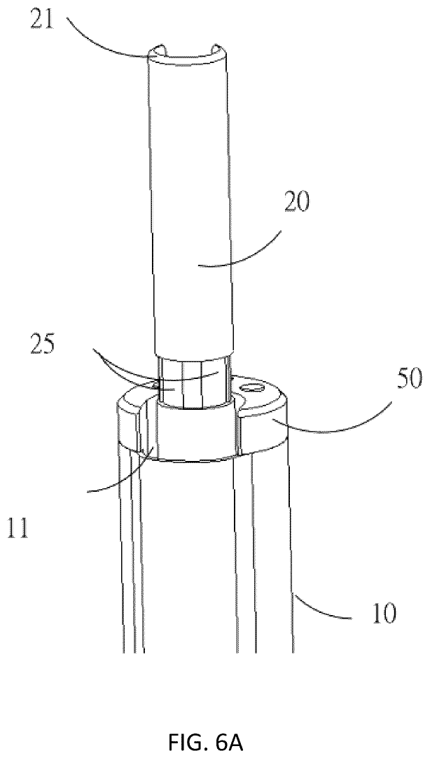

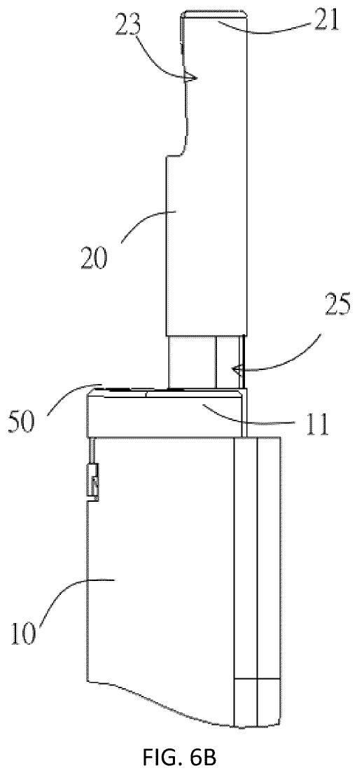

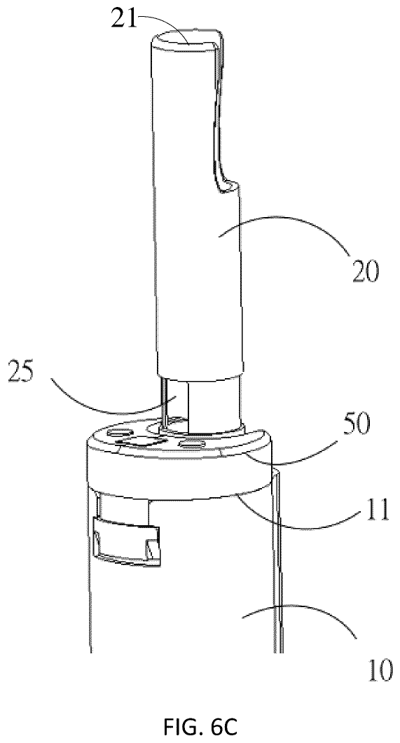

FIGS. 6A, 6B, and 6C illustrate examples of an endoscope device with the second shaft 20 and the first shaft 30 depicted. Referring to FIG. 6A a plurality of proximal openings 25 on second shaft are illustrated. The plurality of openings (e.g., proximal openings, distal openings, or a combination thereof, or openings therebetween) may facilitate similar function and operation of the endoscope device described herein. For example, the second shaft may be configured to oscillate within the first shaft so that either one of the plurality of proximal openings can be aligned and communicated with first shaft proximal opening 33. FIG. 6B illustrates another example of a proximal opening whereby a proximal opening on second shaft can be seen from the side. FIG. 6C is another three-dimensional diagram of the second shaft 20 at a different position. Second shaft 20 is situated that distal opening is covered by first shaft 10, while one of the proximal openings on second shaft 20 is aligned and communicates with the proximal opening of first shaft 33. Suction would then be directed to the proximal openings of the first and second shafts instead of the distal opening, which is now closed.

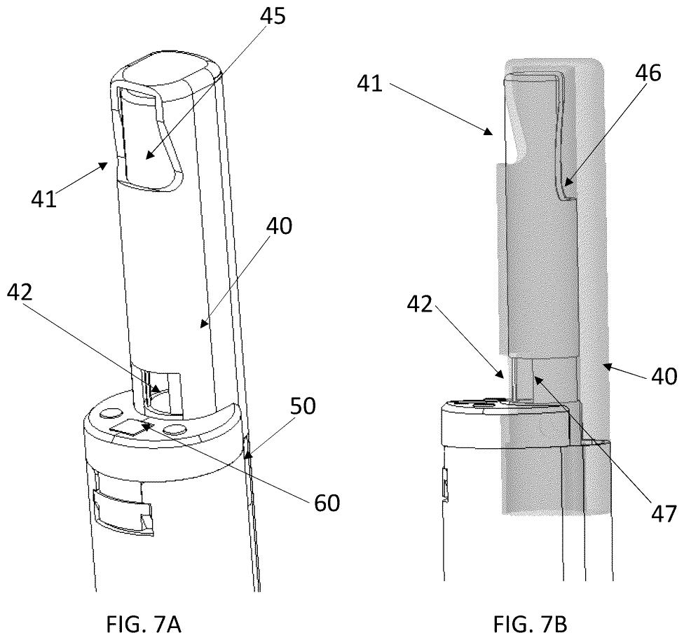

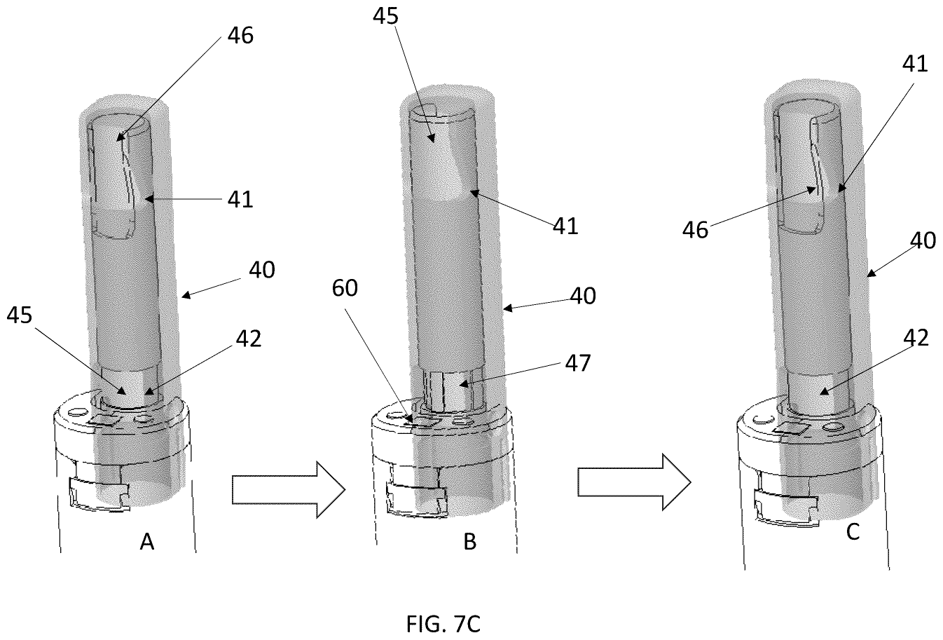

In FIGS. 7A to 7C additional examples of an endoscope device are illustrated. Referring to FIG. 7A the first shaft 40 is illustrated extending from the endoscope distal end 50 having a distal opening 41 and a proximal opening 42 adjacent to the camera module 60. The second shaft 45 is positioned within the first shaft 40. For example, the interior of the first shaft 40 can accommodate the second shaft 45. The distal opening 41 can be configured to direct suction to target tissue such that the target tissue is drawn into the distal opening 41 and cut when the second shaft 45 articulated within the first shaft 40. In FIG. 7A, the second shaft 45 is positioned within the first shaft 40 such that the first shaft distal opening 41 is closed (e.g., obstructed by the second shaft 45). The first shaft proximal opening 42 is shown sufficiently close to the camera module 50 such that when suction is applied and the second shaft proximal opening 47 and first shaft proximal opening 42 are in alignment, suction (e.g., the majority of suction) may be redirected to the aligned proximal openings to remove (e.g., suction) debris from the camera module 50, as shown in FIGS. 7A and 7B.

FIG. 7B illustrates additional detail of the endoscope device of FIG. 7A with the first shaft 40 illustrated in a transparent manner to show the relative position of the second shaft openings. Here, the second shaft distal opening 46 is open to the interior of the first shaft 40. The second shaft proximal opening 47 is visible and aligned with the opening of the first shaft proximal opening 42. Accordingly, the configuration of distal and proximal openings of the first shaft on a single side of the first shaft, while the second shaft distal opening is positioned on a different (e.g., opposite) side of the second shaft relative to the second shaft proximal opening facilitate the alternating alignment (e.g., communication) of openings on the second shaft with openings on the first shaft when the second shaft moves. FIG. 7C illustrates a progression of second shaft 45 movement within the first shaft 40. Here, the alternating alignment as the second shaft 45 is rotated within the first shaft 40 from position A to position B then to position C. The first shaft 40 is illustrated in a transparent manner to highlight the orientation of the second shaft 45 therein. As described herein, rotation is an example of movement of the second shaft, and may include rotation of 360 degrees or less. As described herein, the rotation of the second shaft may be rotation in a first direction (e.g., clockwise) then rotation in the opposite direction (e.g., counterclockwise). Beginning with FIG. 7C, the second shaft 45 is in position whereby the second shaft distal opening 46 and the first shaft distal opening 41 are aligned. Here, suction may be supplied and tissue can be drawn into the aligned distal openings to be cut. For example, suction may be supplied through the second shaft 45 and directed to the proximal opening or distal opening based on which opening set is in alignment. The first shaft proximal opening 42 is obstructed by the second shaft 45 in position A. As the second shaft 45 rotates to position B, the distal openings are no longer in alignment. The first shaft proximal opening is now aligned with the second shaft proximal opening 47 and suction can then be redirected to increase suction at the aligned proximal openings near the camera module 60. Accordingly, the increased suction through the aligned proximal openings can be sufficient to remove debris from the camera module 60. In position C, the second shaft 45 has rotated back whereby the second shaft distal opening 46 and the first shaft distal opening 41 are aligned allowing the process of tissue cutting to be repeated.

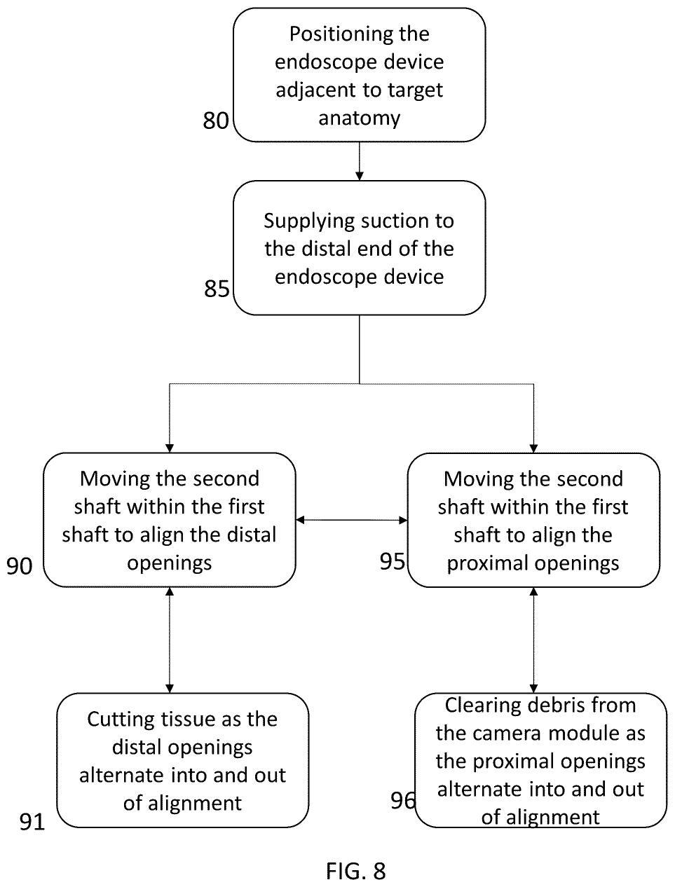

FIG. 8 illustrates a method of using an endoscope device that may begin with positioning the distal segment of the endoscope within a patient at a target location. Step 80 illustrates the positioning of the endoscope within the patient. For example, the endoscope may be navigated through an incision or orifice to a target location for the endoscopic procedure. Then, suction may be supplied at step 85. The suction may be supplied to the second shaft or the first shaft, or the endoscope body, or a dedicated lumen, or a combination thereof. Next, the second shaft is moved within the first shaft at step 90. Movement of the second shaft, as described herein provides for the selective alignment of the distal and proximal openings on the first shaft and the second shaft. The suction is directed to the set of openings that are in alignment at any point in time. Then, at steps 90 and 95, the movement of the second shaft alternates alignment of the proximal and distal openings. When the distal openings are aligned, then the tissue is cut at step 91 when the distal openings transition out of that alignment. Similarly, suction is directed to the proximal openings when they are aligned and debris is cleared from the camera module at step 96.

In some examples described herein, movement of the second shaft may be rotational within the first shaft. For example, the first shaft and the second shaft may have the same of similar central axis and the second shaft may rotate about this axis within the first shaft. Rotation of the second shaft may be facilitated by a motor or other powered device operably coupled to the second shaft. In some examples, rotation or any movement of the second shaft may be based on a user manually articulating the second shaft (e.g., from a proximal end outside of the patient). In some examples, where the second shaft is rotating within the first shaft, the second shaft may be configured to rotate in a first direction (e.g., clockwise or counterclockwise). In some examples, the second shaft may be configured to alternate directions of rotation. For example, the second shaft may first be rotated clockwise then counterclockwise. In some examples, the rotation of the second shaft may be 360 degrees of rotation or more. In some examples, the rotation of the second shaft may be equal to or less than 360 degrees of rotation. For example, the second shaft may rotate in a first direction less than 360 degrees before rotating in the opposite direction. This alternating rotation may be repeated to cut tissue, remove debris from the camera module, or a combination thereof.

In some examples, the second shaft may move laterally within the first shaft. For example, lateral movement of the second shaft may be back-and-forth movement whereby the second shaft transitions from a proximal position to a distal position within the first shaft. The distance between the proximal position of the second shaft and the distal position of the second shaft may be selectively adjusted throughout movement of the second shaft. For example, the second shaft may travel the entire length of the first shaft (e.g., from a proximal end to the distal end. The lateral movement of the second shaft can be configured to alternate alignment of the distal openings and proximal openings on both the first shaft and second shaft, in a linear manner as compared to the rotational manner described with the second shaft rotational movement.

In some examples, the second shaft may move in an oscillating manner, reciprocating manner, or a combination thereof. In some examples, the second shaft may be able to transition between a rotational movement and a lateral movement.

In some examples, the suction supplied to the distal end of the endoscope device (e.g., to the distal and/or proximal openings) may be a constant amount of suction. Accordingly, the alignment and positioning of the openings on the first shaft and second shaft can be configured to direct and control the amount of suction supplied to either the proximal openings or the distal openings or both. For example, alignment of larger openings (e.g., distal openings), may provide increased suction thereto while the proximal openings are not in alignment. For example, when two larger openings align, tissue would be sucked in; when the two small opening align, any stain/liquid in front of camera lens would be sucked in.

In some examples, the debris to be removed by the endoscope device described herein may be tissue (e.g., solid tissue, liquid tissue, or a combination thereof), fluid (e.g., saline, etc.), gasses, biological material, non-biological material, combinations thereof or any other substance that may obstruct the camera module before, during or after use of the endoscope device.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An endoscope, comprising:
a first shaft extending from a distal end of an endoscope body, the first shaft comprising a distal opening and a proximal opening;
a second shaft extending through an interior of the first shaft, the second shaft comprising a distal opening and a proximal opening; and
an image capture module in operable communication with the endoscope body distal end, the endoscope configured to clear debris from the image capture module with suction when the second shaft proximal opening and first shaft proximal opening are in alignment.

2. The endoscope of claim 1, wherein the second shaft distal end is configured to cut tissue extending through the first shaft distal opening.

3. The endoscope of claim 1, wherein the endoscope is configured to alternate suction between the second shaft distal opening and the second shaft proximal opening.

4. The endoscope of claim 1, wherein the second shaft distal opening comprises a perimeter configured to cut tissue extending into the second shaft.

5. The endoscope of claim 4, wherein the second shaft distal opening comprises an inclined perimeter from a proximal side of the second shaft distal opening to a distal side of the proximal shaft distal opening.

6. The endoscope of claim 1, wherein the first shaft distal opening and second shaft distal opening are configured to be in alignment when the first shaft proximal opening and second shaft proximal opening are mis-aligned.

7. The endoscope of claim 1, wherein the first shaft distal opening and second shaft distal opening are configured to be mis-aligned when the first shaft proximal opening and second shaft proximal opening are aligned.

8. The endoscope of claim 1, wherein the first shaft comprises a closed distal end.

9. The endoscope of claim 1, wherein the second shaft is configured to rotate in the first shaft.

10. The endoscope of claim 1, wherein the second shaft comprises a lumen configured to direct suction supplied to the second shaft distal opening and second shaft proximal opening.

11. The endoscope of claim 1, wherein the second shaft is configured to oscillate in the first shaft.

12. The endoscope of claim 11, wherein the second shaft distal opening is the distal end of the second shaft.

13. The endoscope of claim 11, wherein the second shaft is configured to oscillate between a distal position and a proximal position.

14. The endoscope of claim 1, wherein the second shaft proximal opening is positioned on a first side of the second shaft, and the second shaft distal opening is positioned on a second side of the second shaft opposite the first side.

15. The endoscope of claim 1, wherein the second shaft distal opening and second shaft proximal opening are positioned on the same side of the second shaft.

16. The endoscope of claim 1, wherein the second shaft is configured to reciprocate within the first shaft.

17. The endoscope of claim 1, wherein the second shaft is configured to alternate rotation from a first direction to a second direction.

18. The endoscope of claim 1, wherein the first shaft distal opening is positioned on a side of the first shaft, and wherein the second shaft distal opening defines a perimeter of a distal end of the second shaft.

19. An endoscope, comprising:
an endoscope body with a distal end comprising an image capture module;
an outer tube extending distally from the endoscope body, the outer tube comprising a proximal opening and a distal opening; and
an inner tube within the outer tube, the inner tube comprising a proximal opening configured to direct suction to the outer tube distal opening when aligned with the inner tube proximal opening.

20. An endoscope image module clearing system, comprising:
a first shaft extending distally from an endoscope body, the first shaft comprising a distal opening and a proximal opening positioned proximal to an image capture module in a distal end of the endoscope body; and
a second shaft configured to oscillate within the first shaft, the second shaft comprising a distal opening configured to cut tissue, and a proximal opening configured to direct suction to the first shaft proximal opening when aligned with the second shaft proximal opening.

21. An endoscope image module clearing system, comprising:
an inner shaft configured to rotate within an outer shaft, the inner shaft and outer shaft extending from a distal end of an endoscope body; and
an image capture module locatable at a distal end of the endoscope body, wherein the outer shaft comprises an opening adjacent to the image capture module, wherein the inner shaft comprises a proximal opening configured to be selectively aligned with the outer shaft proximal opening to direct suction to the image capture module.

22. A method of cleaning an image capture module of an endoscope, the method comprising:
moving an inner shaft extending distally from an endoscope body, the inner shaft locatable within an interior of an outer shaft,
aligning a proximal opening of the inner shaft with a proximal opening of the outer shaft,
supplying suction from the inner shaft through the inner shaft proximal opening and the outer shaft proximal opening, wherein the suction is configured to clear debris/liquid from the image capture module.

23. A method of cleaning an image capture module of an endoscope during an endoscopic procedure, the method comprising:
oscillating an inner shaft extending distally from an endoscope body, the inner shaft locatable within an interior of an outer shaft;
suctioning tissue into the outer shaft through an outer shaft distal opening;
cutting the tissue with edges of the inner shaft distal opening when the inner shaft distal opening moves relative to the outer shaft distal opening; and
supplying suction from the inner shaft, when a proximal opening of the inner shaft is aligned with a proximal opening of the outer shaft, wherein the supplied suction is configured to clear debris/liquid from the image capture module.

24. A method of cleaning an image capture module of an endoscope during an endoscopic procedure, the method comprising:
reciprocating an inner shaft extending distally from an endoscope body, the inner shaft locatable within an interior of an outer shaft;
alternating alignment of an inner shaft proximal opening with an outer shaft proximal opening; and
supplying suction from the inner shaft proximal opening to the outer shaft proximal opening, when the inner shaft proximal opening is aligned with the outer shaft proximal opening, wherein the suction is configured to clear debris from an image capture module.
